(19) United States
(12) Patent Application Publication
(10) Pub. No.: US 2002/0052509 A1
(43) Pub. Date: May 2, 2002

(54) Calcilytic compounds and method of use

(75) Inventors: Pradip Kumar Bhatnagar, Exton, PA (US); James Francis Callahan, Philadelphia, PA (US); Eric G. Del Mar, Salt Lake City, UT (US); Maria Amparo Lago, Audubon, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/005,490

(22) Filed: Dec. 4, 2001

Related U.S. Application Data
(63) Continuation of application No. 09/647,794, filed on Oct. 5, 2000.
Application No. 09/647,794 is a 371 of international application No. PCT/US99/07760, filed on Apr. 8, 1999.
(60) Provisional application No. 60/081,087, filed on Apr. 8, 1998.

(51) Int. Cl. C07D 333/20; C 07D 3 7/34; C07D 213/54; C07D 211/82; C07D 207/46

(52) U.S. Cl. 546/329; 546/229; 548/566; 549/74; 549/492

(57) ABSTRACT

Calcilytic compounds and compositions and their use in treating abnormal bone or mineral homeostasis.

### FIELD OF INVENTION

The present invention relates to novel calcilytic compounds, pharmaceutical compositions containing these compounds and their use as calcium receptor antagonists.

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evoked by extracellular Ca²⁺. Calcilytics are useful as lead molecules in the discovery, development, design, modification and/or construction of useful calcium modulators which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

### SUMMARY OF THE INVENTION

The present invention comprises novel calcium receptor antagonists represented by Formula (I) hereinbelow and their us in the treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

The present invention further provides a method for antagonizing calcium receptors in an animal, including humans, which comprises administering to an animal in need thereof an effective amount of a compound of Formula (I), indicated hereinbelow.

The present invention further provides a method for increasing serum parathyroid levels in an animal, including humans, which comprises administering to an animal in need thereof an effective amount of a compound of Formula (I), indicated hereinbelow.

### DETAILED DESCRIPTION OF THE INVENTION

The compounds of the present invention are selected from Formula (I) hereinbelow:

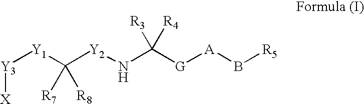

wherein:

Y₁ is a covalent bond, alkylene or alkenylene of up to 4 carbon atoms, unsubstituted or substituted by C₁₋₄ alkyl or O;

Y₂ is methylene, unsubstituted or substituted by C₁₋₄ alkyl or haloalkyl;

Y₃ is covalent bond or O, S, N—R^{IV} or C₁₋₄ alkylene-O, C₁₋₄ alkylene-S, C₁₋₄ alkylene-N—R^{IV};

R^{IV} is selected from the group consisting of H, C₁₋₄ alkyl, C₃₋₆ cycloalkyl;

R₃ and R₄ are, independently, methyl or ethyl, or, together, form cyclopropyl;

R₅ is heteroaryl or fused heteroaryl; wherein the hetero-ring contains N, O or S, and is aromatic, dihydro or tetrahydro, unsubstituted or substituted with any substituents being selected from the group consisting of OH, OCH₃, CH(CH₃)₂, halogen, C₁₋₄ alkyl, C₁₋₄ alkoxy, C₃₋₆ cycloalkyl, OSO₂R^{IV}, CN, NO₂, OCF₃, CF₃, CH₂CF₃, (CH₂)ₙCO₂H, (CH₂)ₙCO₂R^{IV}, and O—(CH₂), CO₂R^{IV};

n is an integer from 0 to 3;

G is a covalent bond, CHR₆ or C—R₆ , wherein R₆ is H, OH or O (forming a ketone);

R₇ is H, OH, or O-C₁₋₄ alkyl;

R₈ is H or C₁₋₄ alkyl; or R₇ and R₈ together form a ketone;

A and B are, independently, selected from the group consisting of a bond, CH₂,

NH, O, S and C═O, provided that either A or B is selected from CH₂ and NH; or A and B together form a bond; or the A-B moiety is represented by CH═CH or C≡C;

X is selected from sub formulas (Ia) to (Ie) hereinbelow:

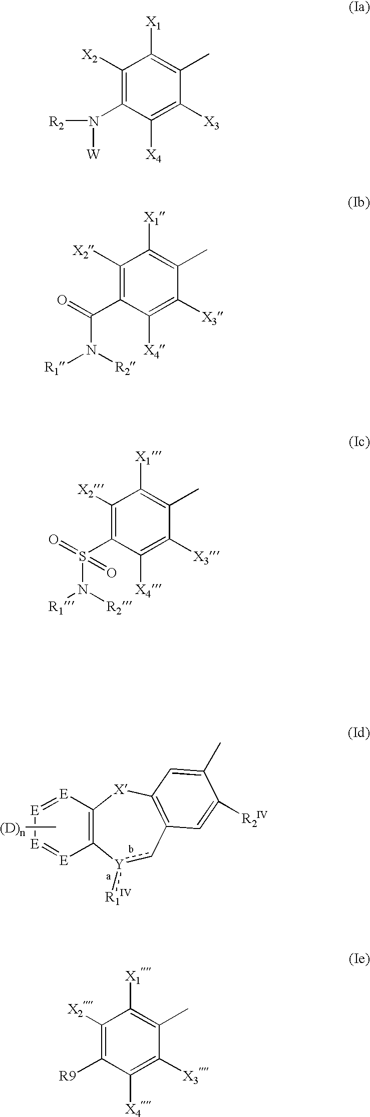

wherein

W is selected from the group consisting of R₁, SO₂R₁, C(O)R₁, SO₂NR₁R₁′, C(O)NR₁R₁′, C(O)OR₁, SO₃R₁′, wherein R₁ and R₁′ are independently selected from the group consisting of hydrogen, C₁₋₄ alkyl, C₃₋₆ cycloalkyl, C₂₋₅ alkenyl, C₂₋₅ alkynyl, heterocycloalkyl, aryl and aryl C₁₋₄ alkyl; or R₁ and R₁′ together form a 3 to 7 membered optionally substituted heterocyclic ring; wherein any substituents are selected from the group consisting of CN, aryl, CO₂R, CO₂NHR, OH, OR, NH₂, halo, CF₃, OCF₃ and NO₂; wherein R represents C₁₋₄ alkyl, or C₃₋₆ cycloalkyl;

X₁ is selected from the group consisting of CN, NO₂, Cl, F, Br, I, H, R′, OR′, CF₃, OCF₃ and OSO₂R′, wherein R′ represents C₁₋₄ alkyl, or C₃₋₆ cycloalkyl;

X₂ , X₃ and X₄ are, independently, selected from the group consisting of CN, NO₂, Cl, F, Br, I, H, R″, OR″, CF₃, OCF₃ and OSO₂R″, provided that either X₁ or X₃ is H, wherein R″ is C₁₋₄ alkyl or haloalkyl; or X₁ and X₂ together form an aryl or heteroaryl ring, substituted or unsubstituted; wherein the heteroatom is selected from N, S and O; and any substituents are selected from the group consisting of halo, C₁₋₄ alkyl, OCF₃, CF₃, OMe, CN, OSO₂R′ and NO₂; or X₃ and X₄ independently represent C(O)R₁; and

R₂ is selected from the group consisting of hydrogen, C₁₋₄ alkyl, C₃₋₆ cycloalkyl, C₂₋₅ alkenyl, C₂₋₅ alkynyl, heterocycloalkyl aryl and aryl-C₁₋₄ alkyl;

X₁″ is selected from the group consisting of CN, NO₂, Cl, F, Br, I, H, R, OR, CF₃, OCF₃ and OSO₂R, wherein R represents C₁₋₄ alkyl, or C₃₋₆ cycloalkyl;

X₂″, X₃″ and X₄″ are, independently, selected from the group consisting of CN, NO₂, Cl, F, Br, I, H, R′, OR′, CF₃, OCF₃ and OSO₂R′, provided that either X″₁ or X″₃ is H, wherein R′ is C₁₋₄ alkyl or haloalkyl; or X₁″ and X₂″ together form an aryl or heteroaryl ring, substituted or unsubstituted; wherein the heteroatom is selected from N, S and O and any substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;

or X₃″ and X₄″ independently represent C(O)R₁; and

R₁″ and R₂″ are, independently, selected from the group consisting of hydrogen, C₁₋₄ alkyl, C₃₋₆ cycloalkyl, C₂₋₅ alkenyl, C₂₋₅ alkynyl, heterocycloalkyl and aryl; or R₁″ and R₂″ together form a 3 to 7 membered optionally substituted heterocyclic ring; wherein any substituents are selected from the group consisting of CN, aryl, CO₂R″, CO₂NHR″, OH, OR″, NH₂, halo, CF₃, OCF₃ and NO₂;

wherein R″ represents C₁₋₄ alkyl, or C₃₋₆ cycloalkyl;

X₁′″ is selected from the group consisting of CN, NO₂, Cl, F, Br, I, H, R, OR, CF₃, OCF₃ and OSO₂R, wherein R represents C₁₋₄ alkyl, or C₃₋₆ cycloalkyl;

X₂′″, X₃′″, and X₄′″ are, independently, selected from the group consisting of CN, NO₂, Cl, F, Br, I, H, R′, OR′, CF₃, OCF₃ and OSO₂R′, provided that either X′″₁ or X′″₃ is H, wherein R′ is C₁₋₄ alkyl or haloalkyl;

or X₁′″ and X₂′″ together form an aryl or heteroaryl ring, substituted or unsubstituted; wherein the heteroatom is selected from N, S and O and the substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;

or X₃″′ and X₄″′ independently represent C(O)R₁;

R₁′″ and R₂′″ are, independently, selected from the group consisting of hydrogen, C₁₋₄ alkyl, C₃₋₆ cycloalkyl, C₂₋₅ alkenyl, C₂₋₅ alkynyl, heterocycloalkyl and aryl;

or R₁′″ and R₂″′ together form a 3 to 7 membered optionally substituted heterocyclic ring; wherein the substituents are selected from the group consisting of CN, aryl, CO₂R″, CO₂NHR″, OH, OR″, NH₂, halo, CF₃, OCF₃ and NO₂; wherein R″ represents C₁₋₄ alkyl, or C₃₋₆ cycloalkyl;

D is selected from the group consisting of H, CN, NO₂, Cl, F, Br, I, R, OR, SR, CF₃, OCF₃ and OSO₂R, wherein R represents C₁₋₄ alkyl, C₃₋₆ cycloalkyl, or C₁₋₁₀aryl or heteroaryl wherein the heteroatom is selected from N, S and O and substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;

n is the integer 1 or 2;

each E is independently C or N, provided that no more than two E moieties are N;

further provided that when n is 2, each E is C;

a and b are optionally present bonds;

R₁^{IV} is selected from the group consisting of (CH₂)ₙCO₂R′, (CH₂)ₙCO₂H, (CH₂)ₙCONR′₂, (CH₂)ₙCH₂OR′, OR′, SR′, CN, NO₂, Cl, F, Br, I, H, CF₃, OCF₃, OSO₂R′, R′ and H; wherein R′ represents C₁₋₄ alkyl, or C₃₋₆ cycloalkyl; or R₁^{IV} is O, forming a ketone such that Y R₁^{IV} represents —C═O;

R₂^{IV} is selected from the group consisting of hydrogen, CN, NO₂ Cl, F, Br, I, H, R″, OR″, CF₃, OCF₃, and OSO₂R″; wherein R″ represents C₁₋₄ alkyl, or C₃₋₆ cycloalkyl.

Y is selected from the group consisting of C, CH, O, N and S; provided that when Y is S, R₁^{IV} is O or not present; further provided that when Y is O, R₁^{IV} is not present;

X′ is selected from the group consisting of CH₂, NH, O and S.

R₉ is selected from the group consisting of O-alkyl, O—CH₂-aryl, and O-aryl;

X₁″″ is selected from the group consisting of CN, NO₂, Cl, F, Br, I, H, R, OR, CF₃, OCF₃ and OSO₂R, wherein R represents C₁₋₄ alkyl, or C₃₋₆ cycloalkyl;

X₂″″, X₃″″, and X₄″″ are, independently, selected from the group consisting of CN, NO₂, Cl, F, Br, I, H, R′, OR′, CF₃, OCF₃ and OSO₂R′, provided that either X″″₁ or X″″₃ is H, wherein R′ is C₁₋₄ alkyl or haloalkyl;

or X₁″″ and X₂″″ together form an aryl or heteroaryl ring, substituted or unsubstituted; wherein the heteroatom is selected from N, S and O and the substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;

or X₂″″ and X₄″″ independently represent C(O)R₁;

and pharmaceutically acceptable salts and complexes thereof.

Preferably, the compounds of the present invention have a structure according to Formula (II):

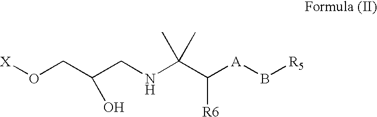

wherein:

R₅ is heteroaryl or fused heteroaryl; wherein the hetero-ring contains N, O or S, and is aromatic, dihydro or tetrahydro, unsubstituted or substituted with any substituents being selected from the group consisting of OH, OCH₃, CH(CH₃)₂, halogen, C₁₋₄ alkyl, C₁₋₄ alkoxy, C₃₋₆ cycloalkyl, OSO₂R^{IV}, CN, NO₂, OCF₃, CF₃, CH₂CF₃, (CH₂)ₙCO₂H, (CH)₂)ₙCO₂R^{IV}, and O—(CH₂)ₙCO₂R^{IV}; and

A and B are, independently, selected from the group consisting of a bond, CH₂, NH, O, S and C═O, provided that either A or B is selected from CH₂ and NH; or A and B together form a bond; or the A-B moiety is represented by CH═CH or C≡C.

More preferably, R₅ is heteroaryl or fused heteroaryl, wherein the hetero-ring contains N, O or S and is aromatic, dihydro or tetrahydro, unsubstituted or substituted with any substituents being selected from the group consisting of OCH₃, halogen, C₁₋₄ alkyl, CN, NO₂, OCF₃, CF₃, CH₂CF₃;

R₆ is H; and

A and B are, independently, selected from the group consisting of a bond, CH₂, NH, O, S and C═O, provided that either A or B is selected from CH₂ and NH, or A and B together form a bond.

Most preferably, R₅ is heteroaryl or fused heteroaryl, wherein the hetero-ring contains N, O or S and is aromatic, dihydro or tetrahydro, unsubstituted or substituted with any substituents being selected from the group consisting of OCH₃, halogen, C₁₋₄ alkyl, CN, NO₂, OCF₃, CF₃, CH₂CF₃;

R₆ is H; and

A and B are, independently, selected from the group consisting of a bond, CH₂, O, or A and B together form a bond.

In sub-formula (Ia), preferably, X₁ is selected from the group consisting of CN, NO₂, Cl, F, Br, I and H. Preferably, X₂, X₃ and X₄ are, independently, selected from the group consisting of Cl, F, Br, I and H, provided X₁ and X₃ is H. Preferably, R₁, R₁′ and R₂ are, independently, selected from the group consisting of C₁₋₄ alkyl, C₃₋₆ cycloalkyl, heterocycloalkyl, aryl or arylalkyl.

In sub-formula (Ia), more preferably, R₁, R₁′ and R₂ are, independently, H, alkyl, or aryl. More preferably, X₁ is selected from the group consisting of CN, NO₂, Cl, F, Br, I and H. More preferably, X₂, X₃ and X₄ are, independently, selected from the group consisting of Cl, F, Br, I and H provided X₁ and X₃ is H.

In sub-formula (Ia), more preferably still, R₁, R₁′ and R₂ are, independently, C₁₋₄ alkyl, or aryl. More preferably still, X₁ is CN, NO₂, or Cl. More preferably still, X₂ is Cl, F or H. More preferably still, X₃ and X₄ are H.

In sub-formula (Ia), most preferably, X₁ is CN, or NO₂. Most preferably, X₂ is Cl.

In sub-formula (Ib), preferably, X₁″ is selected from the group consisting of CN, NO₂, Cl, F, Br, I and H. Preferably, X₂″, X₃″ and X₄″ are, independently, selected from the group consisting of Cl, F, Br, I and H. Preferably, R₁″ and R₂″ are, independently, selected from the group consisting of C₁₋₄ alkyl, C₃₋₆ cycloalkyl, heterocycloalkyl or aryl; or R₁″ and R₂″ together form an optionally substituted 3-7 membered ring, optionally containing an additional heteroatom selected from O, S, and N.

In sub-formula (Ib), more preferably, R₁″ and R₂″ are, independently, H, C₁₋₄ alkyl, or aryl; or R₁″ and R₂″ together form an optionally substituted 4-7 membered ring, optionally containing a heteroatom selected from O, S, and N. More preferably, X₁″ is selected from the group consisting of CN, NO₂, Cl, F, Br, I and H. More preferably, X₂″ is selected from the group consisting of Cl, F, Br, I and H.

In sub-formula (Ib), more preferably still, R₁″ and R₂″ are, independently, C₁₋₄ alkyl, or aryl; or R₁″ and R₂″ together form a 4-7 membered ring as described hereinabove. More preferably still, X₁″ is CN, NO₂, or Cl. More preferably still, X₂″ is Cl, F or H.

In sub-formula (Ib), most preferably, R₁″ and R₂″ together form a 4-7 membered ring as described hereinabove. Most preferably, X₁″ is CN, or NO₂. Most preferably, X₂″ is Cl.

In sub-formula (Ic), preferably, X₁′″ is selected from the group consisting of CN, NO₂, Cl, F, Br, I and H. Preferably, X₂′″, X₃′″ and X₄′″ are, independently, selected from the group consisting of Cl, F, Br, I and H, provided either X₁″′ or X₃″′ is H. Preferably, R₁′″ and R₂″′ are, independently, selected from the group consisting of C₁₋₄ alkyl, C₃₋₆ cycloalkyl, heterocycloalkyl or aryl; or R₁′″ and R₂′″ together form an optionally substituted 3-7 membered ring, optionally containing an additional heteroatom selected from O, S, and N.

In sub-formula (Ic), more preferably, R₁′″, and R₂″′ are, independently, H, C₁₋₄ alkyl, or aryl; or R₁′″ and R₂″′ together form an optionally substituted 4-7 membered ring, optionally containing a heteroatom selected from O, S, and N. More preferably, X₁′″ is selected from the group consisting of CN, NO₂, Cl, F, Br, I and H. More preferably, X₂′″, X₃″′ and X₄′″are, independently, selected from the group consisting of Cl, F, Br, I and H provided either X₁″′ or X₃″′ is H.

In sub-formula (Ic), more preferably still, R₁′″ and R₂′″ are, independently, C₁₋₄ alkyl, or aryl; or R₁″′ and R₂″ together form a 4-7 membered ring as described hereinabove. More preferably still, X₁′″ is CN, NO₂, or Cl. More preferably still, X₂′″ is Cl or H. More preferably still, X₃″′ and X₄′″ are H.

In sub-formula (Ic), most preferably, R₁″′ and R₂″′ together form a 4-7 membered ring as described hereinabove. Most preferably, X₁′″ is CN or NO₂. Most preferably, X₂″′ is Cl.

In sub-formula (Id), preferably, each D is selected from the group consisting of F, Br, Cl, I, R, OR, SR, and H. Preferably, R₁″′ is selected from the group consisting of (CH₂)ₙCO₂R′, (CH₂)ₙCO₂H, (CH₂)ₙCONR′₂, (CH₂)ₙCH₂OR′, OR′, SR′, R′ and H; wherein R′ is as R hereinabove; or R₁^{IV} is O, forming a ketone such that Y R₁^{IV} represents —C═O. Preferably, R₂^{IV} is selected from the group consisting of hydrogen, CN, NO₂, Cl, Br, F and I;

In sub-formula (Id), more preferably, n is O. More preferably, each E is C. More preferably, X′ is CH₂, O, or NH. More preferably, Y is C or N. More preferably, R₁^{IV} is CH₂CO₂R′, SR′, or O forming a ketone.

In subformula (Id), more preferably still, X′ is CH₂ or O. More preferably still, R₁^{IV} is CH₂CO₂R′ or SR′. More preferably still, R₂^{IV} is H, CN, or NO₂.

In subformula (Id), most preferably, X′ is CH₂. Most preferably, Y is C. Most preferably, R₂^{IV} is CN or NO₂.

In subformula (Ie), preferably R₉ is selected from the group consisting of O—(CH₂)ₙ-aryl, and O-aryl;

X₁″″ is selected from the group consisting of CN, NO₂, Cl, F, Br, H, R, and OSO₂R, wherein R represents C₁₋₄ alkyl, or C₃₋₆ cycloalkyl;

X₂″″, X₃″″, and X₄″″ are, independently, selected from the group consisting of CN, NO₂, Cl, F, Br, H, and OSO₂R, provided that either X″″₁ or X″″₃ is H, wherein R′ is C₁₋₄ alkyl or haloalkyl;

or X₁″″ and X₂″″ together form an aryl or heteroaryl ring, substituted or unsubstituted; wherein the heteroatom is selected from N, S and O and the substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;

or X₂″″ and X₄″″ independently represent C(O)R₁;

In subformula (Ie), more preferably R₉ is selected from the group consisting of O—(CH₂)ₙ-aryl, and O-aryl;

X₁″″ is selected from the group consisting of CN, NO₂, and Cl X₂″″, X₃″″, and X₄″″ are, independently, selected from the group consisting of Cl, F, and H, provided that either X″″₁ or X″″₃ is H, or X₂″″ and X₄″″ independently represent C(O)R₁;

In subformula (Ie), most preferably R₉ is selected from the group consisting of O—(CH₂)ₙ-aryl, and O-aryl;

X₁″″ is CN or NO₂,

X₂″″ is Cl, X₃″″ and X₄″″ are, independently F, and H.

Preferred heteroaryls useful in the present invention include unsubstituted and substituted quinolines, isoquinolines, benzofurans, dihydrobenzofurans, benzothiophenes, dibydrobenzothiophenes and pyridines.

As used herein “cycloalkyl” refers to optionally substituted 3-7 membered carbocyclic rings wherein any substituents are selected from the group consisting of, F, Cl, Br, I, N(R₁)₂, SR₁ and OR₁, unless otherwise indicated.

As used herein “heterocycloalkyl” refers to optionally substituted 4, 5, 6 or 7 membered heterocyclic rings containing 1 to 2 heteroatoms selected from N, O, and S.

As used herein, “aryl” refers to an optionally substituted aromatic group with at least one ring having a conjugated pi-electron system, containing up to two conjugated or fused ring systems. Aryl includes carbocyclic aryl, and biaryl groups, all of which may be optionally substituted. Preferred aryl include phenyl and naphthyl. More preferred aryl include phenyl. Preferred substituents are selected from the group consisting of halo, C₁₋₄ alkyl, OCF₃, CF₃, OMe, CN, OSO₂ R and NO₂, wherein R represents C₁₋₄ all or C₃₋₆ cycloalkyl.

As used herein, “acyl” refers to C₁₋₄ alkylcarbonyl.

As used herein, “alkenyl” refers to an optionally substituted hydrocarbon group containing at least one carbon-carbon double bond and containing up to 5 carbon atoms joined together. The alkenyl hydrocarbon chain may be straight, branched or cyclic. Any substituents are selected from the group consisting of halo, C₁₋₄ alkyl, OCF₃, CF₃, OMe, CN, OSO₂ R and NO₂, wherein R represents C₁₋₄ alkyl or C₃₋₆ cycloalkyl.

As used herein, “alkynyl” refers to an optionally substituted hydrocarbon group containing at least one carbon-carbon triple bond between the carbon atoms and containing up to 5 carbon atoms joined together. The alkynyl hydrocarbon group may be straight-chained, branched or cyclic. Any substituents are selected from the group consisting of halo, C₁₋₄ alkyl, OCF₃, CF₃, OMe, CN, OSO₂ R and NO₂, wherein R represents C₁₋₄ alkyl or C₃₋₆ cycloalkyl.

The compounds of the present invention may contain one or more asymmetric carbon atoms and may exist in racemic and optically active forms. All of these compounds and diastereomers are contemplated to be within the scope of the present invention.

Preferred compounds of the present invention are selected from the group consisting of:

(R)-N-[2-Hydroxy-3-(3-chloro-2-cyanophenoxy)propyl]-1,1-dimethyl-2-(2,3-dihydrobenzo[b]furan-5yl)ethylamine;

(R)-N-[2-Hydroxy-3-(3-chloro-2-cyanophenoxy)propyl]-1,1-dimethyl-2-(quinolin-3-yl)ethylamine;

(R)-N-[2-Hydroxy-3-(3-chloro-2-cyanophenoxy)propyl]-1,1-dimethyl-2-(quinolin-2-yl)ethylamine;

(R)-N-[2-Hydroxy-3-(3-chloro-2-cyanophenoxy)propyl]-1,1-dimethyl-2-(isoquinolin-3-yl)ethylamine;

(R)-N-[2-Hydroxy-3-(3-chloro-2-cyanophenoxy)propyl]-1,1-dimethyl-4-(2-pyridyl)butylamine;

(R)-N-[2-Hydroxy-3-(3-chloro-2-cyano-4-morpholinosulfonamidophenoxy)propyl]-1,1-dimethyl-4-(2-pyridyl)butylamine;

(R)-N-[2-Hydroxy-3-(3-chloro-2-cyanophenoxy)propyl]-1,1-dimethyl-4-(3-pyridyl)butylamine;

(R)-N-[2-Hydroxy-3-(3-chloro-2-cyano-4-morpholinosulfonamidophenoxy)propyl]-1,1-dimethyl-4-(3-pyridyl)butylamine;

(R)-N-[2-Hydroxy-3-(3-chloro-2-cyanophenoxy)propyl]-1,1dimethyl-4-(4-carbethoxyphenyl)butylamine;

(R)-N-[2-Hydroxy-3-(3-chloro-2-cyanophenoxy)propyl]-1,1-dimethyl-2-(4-ethylpyrid-2-yl)ethylamine;

(R)-N-[2-Hydroxy-3-(3-chloro-2-cyanophenoxy)propyl]-1,1-dimethyl-2-benzamidoethylamine;

(R)-N-[2-Hydroxy-3-(3-chloro-2-cyanophenoxy)propyl]-4-phenylbutylamine;

(R)-N-[2-Hydroxy-3-(3-chloro-2-cyanophenoxy)propyl]-1,1-dimethyl-4-phenylbut-2-ynylamine;

and pharmaceutically acceptable salts and complexes thereof. Preferred salts include hydrochloride and dihydrochloride.

More preferred compounds useful in the present invention include:

(R)-N-[2-Hydroxy-3-(3-chloro-2-cyanophenoxy)propyl]-1,1-dimethyl-2-(2,3-dihydrobenzo[b]furan-5yl)ethylamine;

(R)-N-[2-Hydroxy-3-(3-chloro-2-cyanophenoxy)propyl]-1,1-dimethyl-2-(quinolin-3-yl)ethylamine;

(R)-N-[2-Hydroxy-3-(3-chloro-2-cyanophenoxy)propyl]-1,1-dimethyl-2-(quinolin-2-yl)ethylamine;

(R)-N-[2-Hydroxy-3-(3-chloro-2-cyanophenoxy)propyl]-1,1-dimethyl-2-(isoquinolin-3-yl)ethylamine;

(R)-N-[2-Hydroxy-3-(3-chloro-2-cyanophenoxy)propyl]-1,1-dimethyl-4-(2-pyridyl)butylamine;

(R)-N-[2-Hydroxy-3-(3-chloro-2-cyano-4-morpholinosulfonamidophenoxy)propyl]-1,1-dimethyl-4-(2-pyridyl)butylamine;

(R)-N-[2-Hydroxy-3-(3-chloro-2-cyanophenoxy)propyl]-1,1-dimethyl-4-(3-pyridyl)butylamine;

(R)-N-[2-Hydroxy-3-(3-chloro-2-cyano-4-morpholinosulfonamidophenoxy)propyl]-1,1-dimethyl-4-(3-pyridyl)butylamine;

(R)-N-[2-Hydroxy-3-(3-chloro-2-cyanophenoxy)propyl]-1,1-dimethyl-2-(4-ethylpyrid-2-yl)ethylamine;

(R)-N-[2-Hydroxy-3-(3-chloro-2-cyanophenoxy)propyl]-1,1-dimethyl-2-benzamidoethylamine;

(R)-N-[2-Hydroxy-3-(3-chloro-2-cyanophenoxy)propyl]-4-phenylbutylamine;

and pharmaceutically acceptable salts and complexes thereof.

The most preferred compounds useful in the present invention include:

(R)-N-[2-Hydroxy-3-(3-chloro-2-cyanophenoxy)propyl]-1,1-dimethyl-2-(2,3-dihydrobenzo[b]furan-5yl)ethyl amine;

(R)-N-[2-Hydroxy-3-(3-chloro-2-cyanophenoxy)propyl]-1,1-dimethyl-2-(quinolin-3-yl)ethylamine;

(R)-N-[2-Hydroxy-3-(3-chloro-2-cyanophenoxy)propyl]-1,1-dimethyl-2-(quinolin-2-yl)ethylamine;

(R)-N-[2-Hydroxy-3-(3-chloro-2-cyanophenoxy)propyl]-1,1-dimethyl-2-(isoquinolin-3-yl)ethylamine;

(R)-N-[2-Hydroxy-3-(3-chloro-2-cyanophenoxy)propyl]-1,1-dimethyl-4-(2-pyridyl)butylamine;

(R)-N-[2-Hydroxy-3-(3-chloro-2-cyano-4-morpholinosulfonamidophenoxy)propyl]-1,1-dimethyl-4-(2-pyridyl)butylamine;

(R)-N-[2-Hydroxy-3-(3-chloro-2-cyanophenoxy)propyl]-1,1-dimethyl-4-(3-pyridyl)butylamine;

(R)-N-[2-Hydroxy-3-(3-chloro-2-cyano-4-morpholinosulfonamidophenoxy)propyl]-1,1-dimethyl-4-(3-pyridyl)butylamine;

(R)-N-[2-Hydroxy-3-(3-chloro-2-cyanophenoxy)propyl]-1,1-dimethyl-2-(4-ethylpyrid-2-yl)ethylamine;

and pharmaceutically acceptable salts and complexes thereof.

Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. A preferred salt is a hydrochloride. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamnine, meglumine, procaine, aluminum, calcium, lithium, magnesium, potassium, sodium, ammonium, alkylamine, and zinc, when acidic functional groups, such as carboxylic acid or phenol are present.

The present invention provides compounds of Formula (I) above, which can be prepared using standard techniques. An overall strategy for preparing preferred compounds described herein can be carried out as described in this section. The examples which follow illustrate the synthesis of specific compounds. Using the protocols described herein as a model, one of ordinary skill in the art can readily produce other compounds of the present invention.

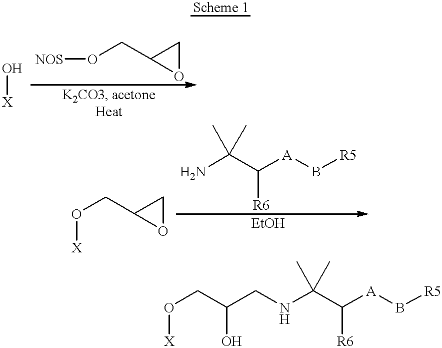

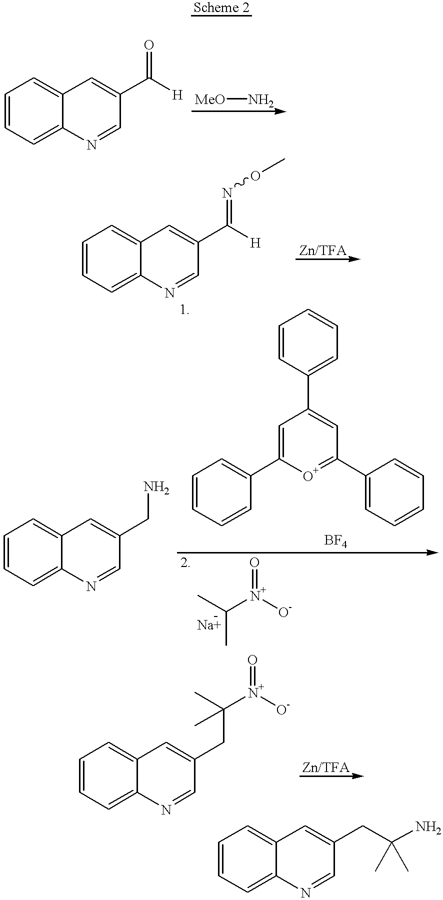

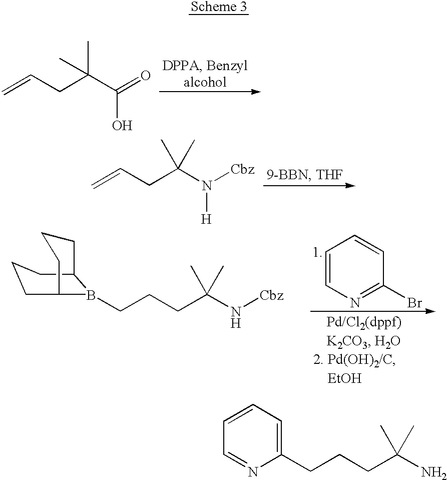

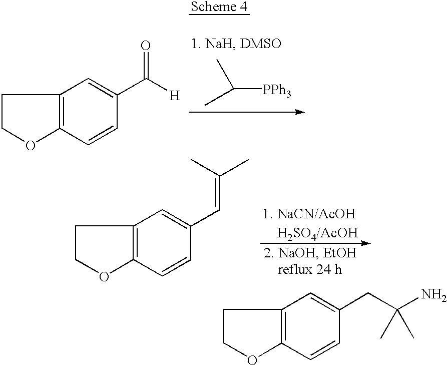

### General Preparation

A general procedure used to synthesize many of the compounds can be carried out as described in Scheme 1: A solution of aryl alcohol in acetone was treated with an appropriate base such as K₂CO₃, heated for 15 min. R-glycidyl nosylate was added and the reaction continued overnight to give the corresponding glycidyl ether (Scheme 1). In the case of an alkyl alcohol, a stronger base, e.g. NaH in DMF was used. This method can also be used for aryl alcohols. A solution of the substituted glycidyl ether and excess amine (typically 1,1-dimethyl-2-(4-methyloxyphenyl)ethylamine) in absolute ethanol, acetonitrile, TBF or any other similar solvent in the presence of a suitable catalyst such as LiClO₄ is stirred overnight at reflux. The product is purified by normal phase chromatography. Hydrochloride salts are prepared by treatment of the corresponding free base with HCl either in gas phase or 4M dioxane solution, or any other standard method.

The synthesis of various corresponding amines is described in Scheme 2, 3, 4 and 5. The synthesis of 3-(2-amino-2-methylpropyl)quinoline illustrates the general procedure to obtain these amines, and it is described in Scheme 2. The reduction of the oxime obtained from 3-quinolinecarboxaldehyde leads to the corresponding benzylic amine. Reaction of the aforementioned amine with 2,4,6-triphenylpyrylium tetrafluoroborate followed by nucleophilic displacement of the pyridinium salt thus formed with the anion of 2-nitropropane, leads to the formation of the corresponding nitro compound which, after reduction, leads to the title compound.

The synthesis of 2-(4-amino-4-methylpentyl)pyridine illustrates the general procedure to obtain pentyl amines, and it is described in Scheme 3. The Curtius rearrangement of 2,2-dimethyl-4-pentenoic acid leads to the corresponding Cbz protected amine. Addition of 9-BBN to the terminal olefin of the protected amine leads to the corresponding boronate. Palladium catalyzed coupling reaction between the boronate and the corresponding aryl bromide (2-bromopyridine in Scheme 3) leads to the formation of the corresponding amine after the removal of the protecting group.

The synthesis of 5-(2-amino-2-methylpropyl)-2,3-dihydrobenzo[b]furan illustrates the general procedure to obtain these amines, and it is described in Scheme 4. Wittig reaction between 2,3-dihydrobenzo[b]furan-5-carboxaldehyde and the anion formed from isopropyltriphenylphosphonium leads to the corresponding olefin. Ritter reaction on the olefin followed by hydrolysis leads to the corresponding amine.

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (●) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. LC/MS/MS was obtained on a Perkin Elmer Sciex API 365 Instrument. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5μ Apex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) Celite® is a filter aid composed of acid-washed diatomaceous silica, and is a registered trademark of Manville Corp., Denver, Colo.

All reagents and solvents were obtained from commercial vendors. Starting materials (e.g., amines and epoxides) were synthesized using standard techniques and procedures.

With appropriate manipulation and protection of any chemical functionality, synthesis of the remaining compounds of Formula (I) is accomplished by methods analogous to those above and to those described in the Experimental section.

In order to use a compound of Formula (I) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered, for example, from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

Another aspect of the present invention describes a method of treating a patient comprising administering to the patient an amount of a present compound sufficient to increase the serum PTH level. Preferably, the method is carried out by administering an amount of the compound effective to cause an increase in duration and/or quantity of serum FM level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In an alternate embodiment of the present invention, the compound administered causes an increase in serum PTH of longer than about twenty-four hours, but the compound is co-administered with an anti-resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

Composition of Formula (I) and their pharmaceutically acceptable salts, which are active when given orally, can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) are demonstrated by the following tests:

(I) Calcium Receptor Inhibitor Assay

Calcilytic activity was measured by determining the IC₅₀ of the test compound for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 Suppl. 1:S483, 1995 (hereby incorporated by reference herein). Intracellular Ca²⁺ increases were elicited by increasing extracellular Ca²⁺ from 1 to 1.75 mM. Intracellular Ca²⁺ was measured using fluo-3, a fluorescent calcium indicator.

The procedure was as follows:

1. Cells were maintained in T-150 flasks in selection media (DMEM supplemented with 10% fetal bovine serum and 200 ug/mL hygromycin B), under 5% CO₂:95% air at 37° C. and were grown up to 90% confluency.

2. The medium was decanted and the cell monolayer was washed twice with phosphate-buffered saline (PBS) kept at 37° C. After the second wash, 6 mL of 0.0₂% EDTA in PBS was added and incubated for 4 minutes at 37° C. Following the incubation, cells were dispersed by gentle agitation.

3. Cells from 2 or 3 flasks were pooled and pelleted (100×g). The cellular pellet was resuspended in 10-15 mL of SPF-PCB+ and pelleted again by centrifugation. This washing was done twice.

Sulfate- and phosphate-free parathyroid cell buffer (SPF-PCB) contains 20 mM Na-Hepes, pH 7.4, 126 mM NaCl, 5 mM KCl, and 1 mM MgCl₂. SPF-PCB was made up and stored at 4° C. On the day of use, SPF-PCB was supplemented with 1 mg/mL of D-glucose and 1 mM CaCl₂ and then split into two fractions. To one fraction, bovine serum albumin (BSA; fraction V, ICN) was added at 5 mg/mL (SPF-PCB+). This buffer was used for washing, loading and maintaining the cells. The BSA-free fraction was used for diluting the cells in the cuvette for measurements of fluorescence.

4. The pellet was resuspended in 10 mL of SPF-PCB+

containing 2.2 uM fluo-3 (Molecular Probes) and incubated at room temperature for 35 minutes.

5. Following the incubation period, the cells were pelleted by centrifugation. The resulting pellet was washed with SPF-PCB+. After this washing, cells were resuspended in SPF-PCB+ at a density of 1-2×106 cells/mL.

6. For recording fluorescent signals, 300 uL of cell suspension were diluted in 1.2 mL of SPF buffer containing 1 mM CaCl₂ and 1 mg/mL of D-glucose. Measurements of fluorescence were performed at 37° C. with consume stirring using a spectrofluorimeter. Excitation and emission wavelengths were measured at 485 and 535 nm, respectively. To calibrate fluorescence signals, digitonin (5 mg/mL in ethanol) was added to obtain Fmax, and the apparent Fmin was determined by adding Tris-EGTA (2.5 M Tris-Base, 0.3 M EGTA). The concentration of intracellular calcium was calculated using the following equation: Intracellular calcium=(F−Fₘᵢₙ/Fₘₐₓ)×K_{d}; where K_{d}=400 nM.

7. To determine the potential calcilytic activity of test compounds, cells were incubated with test compound (or vehicle as a control) for 90 seconds before increasing the concentration of extracellular Ca²⁺ from 1 to 2 mM. Calcilytic compounds were detected by their ability to block, in a concentration-dependent manner, increases in the concentration of intracellular Ca²⁺ elicited by extracellular Ca²⁺.

In general, those compounds having lower IC₅₀ values in the Calcium Receptor Inhibitor Assay are more preferred compounds. Compounds having an IC₅₀ greater than 50 uM were considered to be inactive. Preferred compounds are those having an IC₅₀ of 10 uM or lower, more preferred compounds have an IC₅₀ of 1 uM, and most preferred compounds have an IC₅₀ of 0.1 uM or lower.

(II) Calcium Receptor Binding Assay

HEK 293 4.0-7 cells stably transfected with the Human Parathyroid Calcium Receptor (“HuPCaR”) were scaled up in T180 tissue culture flasks. Plasma membrane is obtained by polytron homogenization or glass douncing in buffer (50 mM Tris-HCl pH 7.4, 1 M EDTA, 3 mM MgCl₂) in the presence of a protease inhibitor cocktail containing 1 uM Leupeptin, 0.04 uM Pepstatin, and 1 mM PMSF. Aliquoted membrane was snap frozen and stored at −80° C. ³H labeled compound was radiolabeled to a radiospecific activity of 44 Ci/mmole and was aliquoted and stored in liquid nitrogen for radiochemical stability.

A typical reaction mixture contains 2 nM ³H compound ((R,R)-N4′-Methoxy-t-3-3′-methyl-1′-ethylphenyl-1-(1-naphthyl)ethylamine), or ³H compound (R)-N-[2-Hydroxy-3-(3-chloro-2-cyanophenoxy)propyl]-1,1-dimethyl-2-(4-methoxyphenyl)ethylamine 4-10 ug membrane in homogenization buffer containing 0.1% gelatin and 10% EtOH in a reaction volume of 0.5 mL. Incubation is performed in 12×75 polyethylene tubes in an ice water bath. To each tube 25 uL of test sample in 100% EtOH is added, followed by 400 uL of cold incubation buffer, and 25 uL of 40 nM ³H-compound in 100% EtOH for a final concentration of 2 nM. The binding reaction is initiated by the addition of 50 uL of 80-200 ug/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting.

The following examples are illustrative, but not limiting of the embodiments of the present invention.

### EXAMPLE 1

### Preparation of (R)-N-[2-Hydroxy-3-(3-chloro-2-cyanophenoxy)propyl]-1,1-dimethyl-2-(2,3-dihydrobenzo[b]furan-5yl)ethylamine Hydrochloride 5-(2-Amino-2-methylpropyl)-2,3-dihydrobenzo[b]furan

Sodium hydride (0.89 g, 37.1 mmole) was added to 45 mL of DMSO and stirred for 30 min at room temperature. Isopropyltriphenylphosphonium iodide (16.05 g, 37.1 mmole) was then added and stirred for 1.5 hours followed by the addition of 2,3-dihydrobenzo[b]furan-5-carboxaldehyde (5.0 g, 33.75 mmole). This mixture was stirred for 18 hours at room temperature then poured into water (300 mL)/conc. HCl (5 mL) and extracted with ether. The crude product was chromatographed on silca gel in 1% MeOH/CHCl₃ to afford 5.1 g (87%) of 5-(2-methylpropenyl)-2,3-dihydrobenzo[b]furan, which was 100% pure by GC-MS. To a 0° C. suspension of sodium cyanide (1.44 g, 29.3 mmole) in 6 mL of acetic acid was slowly added a 0° C. solution of sulfuric acid (3.2 mL) in acetic acid (3.2 mL). After stirring for 45 min at 0° C., 5-(2-methylpropenyl)-2,3-dihydrobenzo[b]furan (5.1 g, 29.3 mmole) was added, and the mixture allowed to warm to room temperature while stirring for 18 hours. The reaction was poured into ice/NaOH and extracted with ether. The ether layer was dried over sodium sulfate, then concentrated in vacuo. The crude amidated product was taken up in EtOH/NaOH and refluxed for 24 hours. The ethanol was removed in vacuo, and the residue taken up in ether and water. The ether layer was separated, dried over sodium sulfate and concentrated in vacuo to yield the crude amine as a dark oil. The product was purified by short-path distillation at reduced pressure.

(R)-N-[2-Hydroxy-3-(3-chloro-2-cyanophenoxy)propyl]-1,1-dimethyl-2-(2,3-dihydrobenzo[b]furan-5yl)ethylamine Hydrochloride

Using previously described methods, (R)-3-chloro-2-cyanophenyl glycidyl ether (0.398 g, 1.9 mmol) and 5-(2-amino-2-methylpropyl)-2,3-dihydrobenzo[b]furan (0.382 g, 2.0 mmol) were used to prepare 100 mg of the title compound as a white solid. ¹H-NMR (CDCl₃).9.65 (1H, m), 8.13 (1H, m), 7.4 (1H, t), 7.05 (2H, d), 6.92 (2H, d), 6.65 (1H, d), 5.7 (1H, d), 4.77 (1H, br m), 4.53 (2H, t), 4.25 (2H, d), 3.4 (2H, m), 3.1 (4H, m), 1.4 (6H, d).

### EXAMPLE 2

### Preparation of (R)-N-[2-Hydroxy-3-(3-chloro-2-cyanophenoxy)propyl]-1,1-dimethyl-2-(quinolin-3-yl)ethylamine Dihydrochloride 3-(2-Amino-2-methylpropyl)quinoline

To a solution of 3-quinolinecarboxaldehyde (10.27 g, 65.3 mmole) in 10 mL of pyridine and 30 mL of ethanol was added methoxylamine hydrochloride (6.0 g, 71.9 mmole). After stirring for 2 hours the solvents were removed under reduced pressure, and the residue taken up in ether and water. The ether layer was separated, dried over sodium sulfate and concentrated in vacuo. The crude oxime (11.91 g, 63.9 mmole) was dissolved in 120 mL of trifluoroacetic acid and treated with zinc powder (13.0 g, 199 mmole). After 10 min, the reaction refluxed spontaneously for a few seconds, and the mixture was stirred for another 3 hours. The mixture was poured into water, and washed with ether. The aqueous layer was then made basic with NaOH, and the amine extracted into ether. The ether layer was separated, dried over sodium sulfate and concentrated in vacuo to yield 8.68 g of 3-(aminomethyl)quinoline. To this amine (8.68 g, 54.9 mmole), dissolved in 200 mL of dichloromethane, was added 2,4,6-triphenylpyrylium tetrafluoroborate (19.56 g, 49.4 mmole), and the reaction stirred at room temperature for 48 hours. The solids were filtered off, and the resulting solution concentrated in vacuo to give 25.4 g (86.3%) of the crude N-(3-quinolinylmethyl)-2,4,6-triphenylpyridinium tetrafluoroborate salt. A solution of this salt (25.4 g, 47.4 mmole) in 100 mL of DMSO was added to the sodium salt of 2-nitropropane (142.1 mmole) (made by adding sodium hydride (3.41 g, 142 mmole) to 50 mL of methanol followed by addition of 2-nitropropane (12.66 g, 142.1 mmole), then removing the methanol in vacuo). The reaction was stirred for 24 hours at 100 C then cooled and diluted with ether and aqueous HCl. The aqueous layer was separated, made basic with NaOH, and extracted with ether. The ether layer was dried over sodium sulfate, and concentrated in vacuo to give after purification on silica gel (in chloroform) 10.7 g (98%) of 3-(2-nitro-2-methylpropyl)quinoline. To this nitro compound (10.7 g, 47.2 mmole) dissolved in 100 mL of trifluoroacetic acid was slowly added zinc powder (9.3 g, 142 mmole). Stirred for 24 hours at room temperature. The reaction mixture was then poured into water and washed with ether. The aqueous layer was separated, made basic with NaOH, and extracted with ether. The ether layer was dried over sodium sulfate, and concentrated in vacuo to give 4.5 g (48%) of 3-(2-amino-2-methylpropyl)quinoline. GC/EI-MS, m/z, (rel. int.) 185 (M⁺-15, 3), 143 (58), 115 (11), 58 (100), 42 (7).

(R)-N-[2-Hydroxy-3-(3-chloro-2-cyanophenoxy)propyl]-1,1-dimethyl-2-(quinolin-3-yl)ethylamine Dihydrochloride

Using previously described methods, (R)-3-chloro-2-cyanophenyl glycidyl ether (0.398 g, 1.9 mmol) and 3-(2-amino-2-methylpropyl)quinoline (0.401 g, 2.0 mmol) were used to prepare 130 mg of the title compound as a white solid. ¹H-NMR (CDCl₃).9.7 (1H, br t), 9.25 (1H, s), 9.0 (2H, br s), 8.42 (1H, d), 8.37 (1H, d), 8.1 (1H, dd), 7.93 (1H, dd), 7.65 (1H, dd), 7.35 (1H, d), 7.28 (1H, d), 4.3 (3H, m), 3.45 (2H, s), 3.3 (2H, m), 2.5 (1H, s), 1.4 (6H, s).

### EXAMPLE 3

### Preparation of (R)-N-[2-Hydroxy-3-(3-chloro-2-cyanophenoxy)propyl]-1,1-dimethyl-2-(quinolin-2-yl)ethylamine Dihydrochloride 2-(2-Amino-2-methylpropyl)quinoline

Using the method of Example 2, supra, 2-(2-amino-2-methylpropyl)quinoline was prepared from quinoline-2-carboxaldehyde. GC/EI-MS, m/z, (rel. int.) 185 (M⁺-15, 5), 143 (42), 115 (13), 58 (100), 42 (6).

(R)-N-[2-Hydroxy-3-(3-chloro-2-cyanophenoxy)propyl]-1,1-dimethyl-2-(quinolin-2-yl)ethylamine Dihydrochloride

Using previously described methods (R)-3-chloro-2-cyanophenyl glycidyl ether (0.21 g, 1.0 mmol) and 2-(2-amino-2-methylpropyl)quinoline (0.24 g, 1.2 mmol) were used to prepare 24 mg of the title compound as a white solid. ¹H-NMR (CDCl₃).9.7 (1H, m), 9.3 (1H, m), 8.55 (1H, d), 8.39 (1H, d), 7.83 (1H, d), 7.7 (2H, m), 7.53 (1H, m), 7.15 (1H, t), 6.75 (2H, m), 4.3 (1H, m), 4.0 (2H, m), 3.65 (2H, dd), 3.15 (2H, m), 1.3 (6H, d).

### EXAMPLE 4

### Preparation of (R)-N-[2-Hydroxy-3-(3-chloro-2-cyanophenoxy)propyl]-1,1-dimethyl-2-(isoquinolin-3-yl)ethylamine Dihydrochloride 3-(2-Amino-2-methylpropyl)isoquinoline

Using the method of Example 2, supra, 3-(2-amino-2-methylpropyl)isoquinoline was prepared from isoquinoline-3-carboxaldehyde. GC/EI-MS, m/z, (rel. int.) 185 (M⁺-15, 7), 144(13), 143(100), 116(9), 115(22), 58 (47), 42 (8).

(R)-N-[2-Hydroxy-3-(3-chloro-2-cyanophenoxy)propyl]-1,1-dimethyl-2-(isoquinolin-3-yl)ethylamine Dihydrochloride

Using previously described methods (R)-3-chloro-2-cyanophenyl glycidyl ether (0.47 g, 2.24 mmol) and 3-(2-amino-2-methylpropyl)isoquinoline (0.49 g, 2.45 mmol) were used to prepare 200 mg of the title compound as a light yellow solid. ¹H-NMR (CDCl₃).9.7 (2H, s on top of m), 9.25 (1H, m), 8.45 (1H, d), 8.27 (1H, s), 8.2 (1H, d), 8.1 (1H, t), 7.9 (1H, t), 7.68 (1H, t), 7.35 (1H, d), 7.25 (1H, d),4.39 (1H, m), 4.3 (2H, s), 3.57 (2H, dd), 3.3 (2H, m), 1.4 (6H, d).

### EXAMPLE 5

### Preparation of (R)-N-[2-Hydroxy-3-(3-chloro-2-cyanophenoxy)propyl]-1,1-dimethyl-4-(2-pyridyl)butylamine Dihydrochloride 4-Benzyloxycarbonylamino-4-methylpent-1-ene

The 2,2-dimethyl-4-pentenoic acid (20.7 g, 162 mmoles) was dissolved in 300 mL of benzyl alcohol followed by addition of triethylamine (17.98 g, 178 mmoles). Diphenyl phosphorylazide (46.67 g, 170 mmoles) was added and the reaction heated to 100° C. overnight under nitrogen. The product was separated from the excess benzyl alcohol by distillation. The product distilled at approx. 130° C. @ 0.01 mm.

9-(4-Benzyloxycarbonylamino-4-methylpentyl)-9-borabicyclo[3.3.1]nonane

To a 0.5 M solution of 9-BBN in THF (100 mL, 50 mmole) was added 4-benzyloxycarbonylaminomethylpent-1-ene (11.67 g, 50 mmole). The reaction was allowed to stand for 24 hours at room temperature. Analysis by GC-MS showed no starting alkene left. The solution, which was approximately 0.439 M in the borane, was used without purification.

2-(4-Amino-4-methylpentyl)pyridine

To 2-bromopyridine (0.948 g, 6 mmole) was added 9-(4-benzyloxycarbonylamino-4-methylpentyl)-9-borabicyclo[3.3.1]nonane (12 mL, 5 mmole of a THF solution) in a nitrogen flushed reaction tube. To this solution was added 0.122 g (0.15 mmole) of [1,1′-bis(diphenylphosphino)ferrocene]dichloropalladium(II) dichloromethane complex (1:1), 1.38 g (10 mmole) of potassium carbonate, and 1.25 mL of water. The reaction was stirred for 18 hours at 65 C, then poured into aqueous NaOH, and extracted with ether. The ether layer was separated, washed with brine, dried over sodium sulfate, and concentrated in vacuo. The crude product was taken up in 20 mL of ethanol to which 1 g of palladium hydroxide on carbon (10%) was added. The mixture was stirred for 18 hours under a hydrogen balloon. The reaction mixture filtered and concentrated in vacuo. The residue was taken up in aqueous HCl, and extracted with ether. The aqueous layer was separated, made basic with NaOH, and extracted with ether. The ether layer was dried over sodium sulfate, and concentrated in vacuo to give 0.63 g of 2-(4-Amino-4-methylpentyl)pyridine.

(R)-N-[2-Hydroxy-3-(3-chloro-2-cyanophenoxy)propyl]-1,1-dimethyl-4-(2-pyridyl)butylamine Dihydrochloride

Using previously described methods (R)-3-chloro-2-cyanophenyl glycidyl ether (0.21 g, 1.0 mmol) and 2-(4-Aminomethylpentyl)pyridine (0.196 g, 1.1 mmol) were used to prepare 19 mg of the tide compound as a light yellow solid. LC/MS/MS* (In-Source ISD technique), m/z, 388 (M⁺), 227, 162, 106.

### EXAMPLE 6

### Preparation of (R)-N-[2-Hydroxy-3-(3-chloro-2-cyano-4-morpholinosulfonamidophenoxypropyl]-1,1-dimethyl-4-(2-pyridyl)butylamine Dihydrochloride

Using the method of Example 5, supra, (R)-3-chloro-2-cyano-4-morpholinosulfonamidophenyl glycidyl ether (0.3 g, 0.83 mmol) and 2-(4-Amino-4-methylpentyl)pyridine (0.156 g, 0.87 mmol) were used to prepare 150 mg of the title compound as an off white solid. ¹H-NMR (CDCl₃)..9.3 (1H, m), 8.8 (2H, d on top of m), 8.55 (1H, t), 8.18 (1H, d), 8.02 (1H, d), 7.95 (1H, dd), 7.5 (1H, d), 4.4 (3H, m), 3.6 (4H, br s), 3.1 (6H, m), 2.5 (2H, s), 1.8 (4H, m), 1.3 (6H, s).

### EXAMPLE 7

### Preparation of (R)-N-[2-Hydroxy-3-(3-chloro-2-cyanophenoxypropyl]-1,1-dimethyl-4-(3-pyridyl)butylamine Dihydrochloride 3-(4-Amino-4-methylpentyl)pyridine

Using the method of Example 5, supra, 0.66 g of 3-(4-Amino-4-methylpentyl)pyridine was prepared, starting with 6 mmoles of 3-bromopyridine.

(R)-N-[2-Hydroxy-3-(3-chloro-2-cyanophenoxy)propyl]-1,1-dimethyl-4-(3-pyridyl)butylamine Dihydrochloride

Using previously described methods, (R)-3-chloro-2-cyanophenyl glycidyl ether (0.21 g, 1.0 mmol) and 3-(4-Amino-4-methylpentyl)pyridine (0.196 g, 1.1 mmol) were used to prepare 25 mg of the title compound as a light yellow glassy solid. LC/MS/MS* (In-Source ISD technique), m/z 388 (M⁺), 227, 162, 106.

### EXAMPLE 8

### Preparation of (R)-N-[2-Hydroxy-3-(3-chloro-2-cyano-4-morpholinosulfonamidophenoxy)propyl]-1,1-dimethyl-4-(3-pyridyl)butylamine Dihydrochloride

Using the method of Example 5, supra, (R)-3-chloro-2-cyano-4-morpholinosulfonamidophenyl glycidyl ether (0.3 g, 0.83 mmol) and 3-(4-Amino-4-methylpentyl)pyridine (0.156 g, 0.87 mmol) were used to prepare 30 mg of the title compound as an off white solid. ¹H-NMR (CDCl₃).9.3 (1H, m), 8.9 (1H, s), 8.8 (2H, d on top of m), 8.56 (1H, d), 8.16(1H, d), 8.04(1H, dd), 7.5 (1H, d), 4.4 (3H, m), 3.6 (4H, br s), 3.1 (4H, m), 2.8 (2H, br s), 2.5 (2H, s), 1.7 (4H, m), 1.3 (6H, s).

### EXAMPLE 9

### Preparation of (R)-N-[2-Hydroxy-3-(3-chloro-2-cyanophenoxy)propyl]-4-phenylbutylamine Hydrochloride

Using previously described methods, (R)-3-chloro-2-cyanophenyl-glycidyl ether (0.21 g, 1.0 mmol) and 4-phenylbutylamine (0.164 g, 1.1 mmol) were used to prepare 250 mg of the title compound as a white solid. ¹H NMR (CDCl₃) d 10.09 (1H, s), 9.38 (1H, s), 9.12 (1H, s), 7.53 (2H, m), 7.19 (7H, m), 6.08 (1H, m), 5.98 1H, m), 4.63 (1H, m), 4.52 (1H, m), 4.23 (3H, m), 3.95 (1H, m), 3.43 (1H, m), 3.20 (1H, m), 3.00 (1H, m), 2.65 (3H, m), 1.86 (2H, m), 1.71 (2H, m).

### EXAMPLE 10

### Preparation of (R)-N-[2-Hydroxy-3-(3-chloro-2-cyanophenoxy)propyl-1,1-dimethyl-4-(2-carbethoxyphenyl)butylamine Hydrochloride Ethyl 2-(4-Amino-4-methypentylbenzoate

To ethyl 2-bromobenzoate (0.504 g, 2.2 mmole) in a nitrogen flushed reaction tube was added 0.049 g (0.06 mmole) of [1,1′-bis(diphenylphosphino)ferrocene]dichloropalladium(II) dichloromethane complex (1:1) dissolved in 2 mL of DMF. To this solution was added 1.3 g (4 mmoles) of cesium carbonate, followed by 9-(4-benzyloxycarbonylamino-4-methylpentyl)-9-borabicyclo[3.3.1]nonane (4.56 mL, 2.0 mmole of a THF solution). The reaction was stirred for 16.5 hours at 50 C, then poured into aqueous NaOH, and extracted with ether. The ether layer was separated, washed with brine, dried over sodium sulfate, and concentrated in vacuo. The crude product was taken up in 10 mL of ethanol to which 0.3 g of palladium hydroxide on carbon (10%) was added. The mixture was stirred for 18 hours under a hydrogen balloon. The reaction mixture was filtered and concentrated in vacuo. The residue was taken up in aqueous HCl, and extracted with ether. The aqueous layer was separated, made basic with NaOH, and extracted with ether. The ether layer was dried over sodium sulfate, and concentrated in vacuo to give crude ethyl 2-(4-amino-4-methylpentyl)benzoate. The crude product was purified by reversed-phase HPLC on a C-18 column using a gradient of 0.1 % HCl to 40% acetonitrile in 0.1 % HCl.

(R)-N-[2-Hydroxy-3-(3-chloro-2-cyanophenoxy)propyl]-1,1-dimethyl-4-(2-carbethoxyphenyl)butylamine Hydrochloride

Using previously described methods, (R)-3-chloro-2-cyanophenyl glycidyl ether (0.21 g, 1.0 mmol) and 1,1-dimethyl-4-(2-carboxyphenyl)butylamine (0.274 g, 1.1 mmol) were used to prepare 260 mg of the title compound as a while solid. ¹H NMR (CDCl₃).9.54 (1H, s), 8.17 (1H, m), 7.85 (1H, dd), 7.43 (2H, m), 7.24 (2H, m), 7.06 (1H, d), 6.97 (1H, d), 6.00 (1H, d), 4.71 (1H, s), 4.33 (2H, q), 4.26 (2H, d), 3.27 (2H, m), 2.60 (2H, m), 1.85 (2H, m), 1.69 (2H, m), 1.46 (6H, s), 1.37 (3H, t).

### EXAMPLE 11

### Preparation of (R)-N-[2-Hydroxy-3-(3-chloro-2-cyanophenoxy)propyl]-1,1-dimethyl-4-(3-carbethoxyphenyl)butylamine Hydrochloride

Using previously described methods, (R)-3-chloro-2-cyanophenyl glycidyl ether (0.21 g, 1.0 mmol) and 1,1-dimethyl-4-(3-carbethoxyphenyl)butylamine (0.274 g, 1.1 mmol) were used to prepare 230 mg of the title compound as a white solid. ¹H NMR (CDCl₃).9.56 (1H, m), 8.21 (1H, m), 7.86 (2H, m), 7.37 (3H, m), 7.07 (1H, d), 6.94 (1H, d), 5.59 (1H, d), 4.70 (1H, m), 4.35 (2H, q), 4.24 (2H, d), 3.25 (2H, m), 2.71 (2H, m), 1.82 (4H, m), 1.49 (6H, s), 1.39 (3H, t).

### EXAMPLE 12

### Preparation of (R)-N-[2-Hydroxy-3-(3-chloro-2-cyanophenoxy)propyl]-1,1-dimethyl-4-(4-carbethoxyphenyl)butylamine Hydrochloride

Using the method of Example 5, supra, (R)-3-chloro-2-cyanophenyl glycidyl ether (0.21 g, 1.0 mmol) and 1,1-dimethyl-4-(4-carbethoxyphenyl)butylamine (0.274 g, 1.1 mmol) were used to prepare 250 mg of the title compound as a white solid. ¹H NMR (CDCl₃).9.56 (1H, m), 8.19 (1H, m), 7.95 (2H, d), 7.43 (1H, ddd), 7.26 (2H, d), 7.07 (1H, d), 6.93 (1H, d), 5.58 (1H, d), 4.69 (1H, m), 4.33 (2H, q), 4.22 (2H, d), 3.23 (2H, m), 2.71 (2H, m), 1.48 (3H, s), 1.47 (3H, s), 1.37 (3H, t).

### EXAMPLE 13

### Preparation of (R)-N-[2-Hydroxy-3-(3-chloro-2-cyanophenoxy)propyl]-1,1-dimethyl-2-(4-ethylpyrid-2-yl)ethylamine Dihydrochloride 1,1-Dimethyl-2-(4-ethylpyrid-2-yl)ethylamine

4 mmoles 5-ethyl-2-methyl pyridine in 4 mL dry ether was treated with 4.32 mmoles of phenyl lithium (1.8 M solution in cyclohexane/ether) at 0° C. After reaction at RT for 1 h the solution was added dropwise to a chilled (ice bath) solution of 2 mmoles isopropylidene-3-nitrobenzene sulfenamide in 2 mL dry ether. After reaction at RT for 1 hr and at reflux for 0.5 hrs the cooled reaction mixture was quenched with 5 mls water. The organic layer was extracted three times with 6 Molar HCl. The pooled HCl extracts were evaporated to an oil, made basic with 10 N NaOH and extracted with ether. The ether was extracted twice with pH 7 Phosphate Buffer, buffer extracts made basic with NaOH and extracted with chloroform. Removal of the chloroform resulted in the title compound in 24% yield.

(R)-N-[2-Hydroxy-3-(3-chloro-2-cyanophenoxy)propyl]-1,1-dimethyl-2-(4-ethylpyrid-2-yl)ethylamine Dihydrochloride

Using previously described methods, (R)-3-chloro-2-cyanophenyl glycidyl ether (0.21 g, 1.0 mmol) and 1,1-Dimethyl-2-(4-ethylpyrid-2-yl)ethylamine (0.25 g, 1.4 mmol) were used to prepare 314 mg of the title compound as a white solid. ¹H NMR (CDCl₃).9.95 (1H, br s), 9.05 (1H, br s), 8.62 (1H,s), 8.27 (1H, br s), 8.02 (1H, br s), 7.44 (1H, t), 7.0 (2H, d), 5.6 (2H, br s), 4.7 (1H, m), 4.34 (2H, br s), 3.9 (2H, br s), 3.53 (2H, br s), 2.86 (2H, q), 1.62 (6H, s), 1.32 (3H, t).

### EXAMPLE 14

### (R)-N-[2-Hydroxy-3-(3-chloro-2-cyanophenoxy)propyl]-1,1-dimethyl-2-benzamidoethylamine Hydrochloride

Using previously described methods (R)-2-chloro-2-cyanophenyl glycidyl ether (0.00 g, 00 mmol) and 1,1-Dimethyl-2-benzamidoethylamine (00 g, 00 mmol) were used to prepare 000 mg of the title compound as a white solid. ¹H-NMR (CDCl₃).9.52 (1H, m), 8.40 (1H, m), 8.20 (1H, s) 7.95 (1H, d), 7.36 (4H, m), 7.02 (1H, d), 6.82 (1H, d), 4.70 (1H, m), 4.16 (2H, m), 3.82 (3H, m), 3.37 (2H, m), 1.51 (3H, s), 1.46(3H, s); ¹³C NMR (CDCl₃)..169.2, 161.3, 137.7, 134.7, 133.1, 131.9, 128.5, 127.8, 122.3, 113.8, 110.8, 103.2, 77.2, 70.8, 65.5, 61.4, 46.4, 44.8, 22.0, 21.6.

### EXAMPLE 15

### (R)-N-[2-Hydroxy-3-(3-chloro-2-cyanophenoxy)propyl]-1,1-dimethyl-4-phenylbut-2-ynylamine Hydrochloride

Using previously described methods, (R)-3-chloro-2-cyanophenyl glycidyl ether (0.00 g, 00 mmol) and 1,1-Dimethyl-4phenylbut-2-ynylamine (00 g, 00 mmol) were used to prepare 000 mg of the title compound as a white solid. ¹H-NMR (CDCl₃)..9.97 (1H, m), 8.76 (1H, m), 7.41 (1H, ddd), 7.30 (3H, m), 7.18 (2H, m), 7.04 (1H, d), 6.92 (1H, d), 5.63 (1H, m), 4.76 (1H, m), 4.23 (2H, m), 3.51 (2H, m), 1.85 (3H, s), 1.83 (3H, s); ¹³C NMR (CDCl₃) 161.4, 137.8, 135.7, 134.5, 128.2, 127.9, 126.8, 122.2, 113.5, 110.9, 103.4, 86.8, 79.0, 70.9, 65.3, 55.5, 46.8, 26.8, 26.7, 24.7.

### EXAMPLE 16

### Preparation of 1,1-dimethyl-2-[(ethyl-4-oxyacetate)-phenyl]ethylamine

A mixture of 1-nitro-1,1-dimethyl-2-(4-hydroxyphenyl]ethane (3.9 g, 0.02 mole), K₂CO₃ (2.76 g, 0.02 mole) and ethylbromoacetate (3.06 g, 0.02 mole) were refluxed in 75 ml of acetone for 18 h. The reaction was cooled to room temperature and filtered. The filtrate was concentrated in vacuo to yield 5.5 g of an oil. This oil was dissolved in 75 ml of EtOH and 1 large spatula-full of washed Raney-nickel was added under argon. The mixture was hydrogenated at room temperature and 55 psi for 18 H. The reaction was filtered and the filtrate concentrated in vacuo to an oil which was filtered through a pad of silver gel eluting with 10% MeOH-CH₂Cl₂ (v/v). The first 200 ml were combined and concentrated in vacuo to yield 3.2 g of a pale yellow oil. MS, m/z 252 (M+H), 503 (2M+H).

Formulations for pharmaceutical use incorporating compounds of the present invention can be prepared in various forms and with numerous excipients. Examples of such formulations are given below.

### EXAMPLE 17

### Inhalant Formulation

A compound of Formula (I) (1 mg to 100 mg) is aerosolized from a metered dose inhaler to deliver the desired amount of drug per use.

EXAMPLE 18
Tablet Formulation

| Tablets/Ingredients | | Per Tablet |
|---|---|---|
| 1. | Active ingredient | 40 mg |
| | (Cmp. of Formula(I)) | |
| 2. | Corn Starch | 20 mg |
| 3. | Alginic acid | 20 mg |
| 4. | Sodium Alginate | 20 mg |
| 5. | Mg stearate | 13 mg |

### Procedure for Tablet Formulation

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 19

### Parenteral Formulation

A pharmaceutical composition for parenteral administration is prepared by dissolving an appropriate amount of a compound of Formula (I) in polyethylene glycol with heating. This solution is then diluted with water for injections (to 100 ml). The solution is then rendered sterile by filtration through a 0.22 micron membrane filter and sealed in sterile containers.

All publications, including but not limited to patents and patent applications cited in this specification are herein incorporated by reference as if each individual publication were specifically and individually indicated to be incorporated by reference as though fully set forth.

What is claimed is:

1. A compound according to Formula (I) hereinbelow: 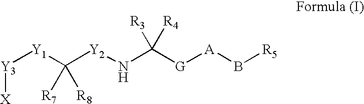
wherein:
Y₁ is a covalent bond, alkylene or alkenylene of up to 4 carbon atoms, unsubstituted or substituted by C₁₋₄ alkyl or O;
Y₂ is methylene, unsubstituted or substituted by C₁₋₄ alkyl or haloalkyl;
Y₃ is covalent bond or selected from the group consisting of O, S, N—R^{IV}, C₁₋₄ alkylene-O, C₁₋₄ alkylene-S, and C₁₋₄ alkylene-N—R^{IV};
R^{IV} is selected from the group consisting of H, C₁₋₄ alkyl, and C₃₋₆ cycloalkyl;
R₃ and R₄ are, independently, methyl or ethyl, or, together, form cyclopropyl;
R₅ is heteroaryl or fused heteroaryl; wherein the hetero-ring contains N, O or S, and is aromatic, dihydro or tetrahydro, unsubstituted or substituted with any substituents being selected from the group consisting of OH, OCH₃, CH(CH₃)₂, halogen, C₁₋₄ alkyl, C₁₋₄ alkoxy, C₃₋₆ cycloalkyl, OSO₂R^{IV}, CN, NO₂, OCF₃, CF₃, CH₂CF₃, (CH₂)ₙCO₂H, (CH₂)ₙCO₂R^{IV}, and O—(CH₂), CO₂R^{IV};
n is an integer from 0 to 3;
G is a covalent bond, CHR₆ or C—R₆, wherein R₆ is H, OH or O (forming a ketone);
R₇ is H, OH, or O-C₁₋₄ alkyl;
R₈ is H or C₁₋₄ alkyl; or R₇ and R₈ together form a ketone;
A and B are, independently, selected from the group consisting of a bond, CH₂, NH, O, S and C═O, provided that either A or B is selected from CH₂ and NH; or A and B together form a bond; or the A-B moiety is represented by CH═CH or C≡C;
X is selected from sub formulas (Ia) to (Ie) hereinbelow: 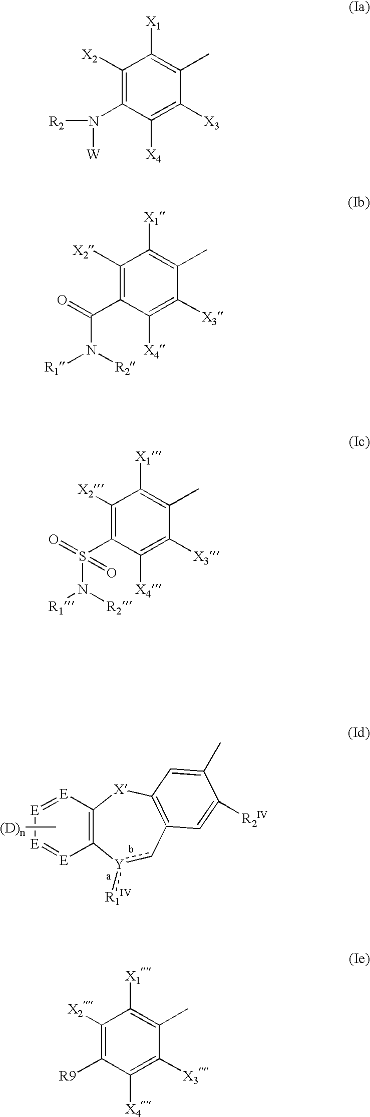
wherein
W is selected from the group consisting of R₁, SO₂R₁, C(O)R₁, SO₂NR₁R₁′, C(O)NR₁R₁′, C(O)OR₁, and SO₃R₁′, wherein R₁ and R₁′ are independently selected from the group consisting of hydrogen, C₁₋₄ alkyl, C₃₋₆ cycloalkyl, C₂₋₅ alkenyl, C₂₋₅ alkynyl, heterocycloalkyl, aryl and aryl C₁₋₄ alkyl; or R₁ and R₁′together form a 3 to 7 membered optionally substituted heterocyclic ring; wherein any substituents are selected from the group consisting of CN, aryl, CO₂R, CO₂NHR, OH, OR, NH₂, halo, CF₃, OCF₃ and NO₂; wherein R represents C₁₋₄ alkyl, or C₃₋₆ cycloalkyl;
X₁ is selected from the group consisting of CN, NO₂, Cl, F, Br, I, H, R′, OR′, CF₃, OCF₃ and OSO₂R′, wherein R′ represents C₁₋₄ alkyl, or C₃₋₆cycloalkyl;
X₂, X₃ and X₄ are, independently selected from the group consisting of CN NO₂, Cl, F, Br, I, H, R″, OR″, CF₃, OCF₃ and OSO₂R, provided that either X₁ or X₃ is H, wherein R″ is C₁₋₄ alkyl or haloalkyl; or X₁ and X₂ together form an aryl or heteroaryl ring, substituted or unsubstituted; wherein the heteroatom is selected from N, S and O; and any substituents are selected from the group consisting of halo, C₁₋₄ alkyl, OCF₃, CF₃, OMe, CN, OSO₂R′ and NO₂; or X₃ and X₄ independently represent C(O)R₁; and
R₂ is selected from the group consisting of hydrogen, C₁₋₄ alkyl, C₃₋₆ cycloalkyl, C₂₋₅ alkenyl, C₂₋₅ alkynyl, heterocycloalkyl aryl and aryl-C₁₋₄ alkyl;
X₁″ is selected from the group consisting of CN, NO₂, Cl, F, Br, I, H, R, OR, CF₃, OCF₃ and OSO₂R, wherein R represents C₁₋₄ alkyl, or C₃₋₆ cycloalkyl;
X₂″, X₃″ and X₄″ are, independently, selected from the group consisting of CN, NO₂, Cl, F, Br, I, H, R′, OR′, CF₃, OCF₃ and OSO₂R′, provided that either X″₁ or X″₃ is H, wherein R′ is C₁₋₄ alkyl or haloalkyl; or X₁″ and X₂″ together form an aryl or heteroaryl ring, substituted or unsubstituted; wherein the heteroatom is selected from N, S and O and any substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;
or X₃″ and X₄″ independently represent C(O)R₁; and
R¹″ and R₂″ are, independently, selected from the group consisting of hydrogen, C₁₋₄ alkyl, C₃₋₆ cycloalkyl, C₂₋₅ alkenyl, C₂₋₅ alkynyl, heterocycloalkyl and aryl; or R₁ ″ and R₂″ together form a 3 to 7 membered optionally substituted heterocyclic ring; wherein any substituents are selected from the group consisting of CN, aryl, CO₂R″, CO₂NHR″, OH, OR″, NH₂, halo, CF₃, OCF₃ and NO₂;
wherein R″ represents C₁₋₄ alkyl, or C₃₋₆ cycloalkyl;
X₁′″ is selected from the group consisting of CN, NO₂, Cl, F, Br, I, H, R, OR, CF₃, OCF₃ and OSO₂R, wherein R represents C₁₋₄ alkyl, or C₃₋₆ cycloalkyl;
X₂′″, X₃′″, and X₄′″ are, independently, selected from the group consisting of CN, NO₂, Cl, F, Br, I, H, R′, OR′, CF₃, OCF₃ and OSO₂R′, provided that either X″′₁ or X′″₃ is H, wherein R′ is C₁₋₄ alkyl or haloalkyl;
or X₁′″ and X₂″′ together form an aryl or heteroaryl ring, substituted or unsubstituted; wherein the heteroatom is selected from N, S and O and the substituents are selected from the group consisting of halo, C₁₋₄alkyl, OCF₃, CF₃, OMe, CN, OSO₂-C₁₋₄alkyl, OSO-₂-C₃₋₆cycloalkyl and NO₂;
or X₃″′ and X₄′″ independently represent C(O)R₁;
R₁′″ and R₂″′ are, independently, selected from the group consisting of hydrogen, C₁₋₄ alkyl, C₃₋₆ cycloalkyl, C₂₋₅ alkenyl, C₂₋₅ alkynyl, heterocycloalkyl and aryl, or R₁″′ and R₂″′ together form a 3 to 7 membered optionally substituted heterocyclic ring; wherein the substituents are selected from the group consisting of CN, aryl, CO₂R″, CO₂NHR″, OH, OR″, NH₂, halo, CF₃, OCF₃ and NO₂; wherein R″ represents C₁₋₄ alkyl, or C₃₋₆ cycloalkyl;
D is selected from the group consisting of H, CN, NO₂, Cl, F, Br, I, R, OR, SR, CF₃, OCF₃ and OSO₂R, wherein R represents C₁₋₄ alkyl, C₃₋₆ cycloalkyl, or C₁₋₁₀aryl or heteroaryl wherein the heteroatom is selected from N, S and O and substituents are selected from the group consisting of halo, C₁₋₄ alkyl, OCF₃, CF₃, OMe, CN, OSO₂-C₁₋₄ alkyl, OSO₂-C₃₋₆cycloalkyl and NO₂;
n is the integer 1 or 2;
each E is independently C or N, provided that no more than two E moieties are N; further provided that when n is 2, each E is C;
a and b are optionally present bonds;
R₁^{IV} is selected from the group consisting of (CH₂)ₙCO₂R′, (CH₂)ₙCO₂H, (CH₂)ₙCONR′₂, (CH₂)ₙCH₂OR′, OR′, SR′, CN, NO₂, Cl, F, Br, I, H, CF₃, OCF₃, OSO₂R′, R′ and H; wherein R′ represents C₁₋₄ alkyl, or C₃₋₆ cycloalkyl; or R₁^{IV} is O, forming a ketone such that Y R₁^{IV} represents —C═O;
R₂^{IV} is selected from the group consisting of hydrogen, CN, NO₂ Cl, F, Br, I, H, R″, OR″, CF₃, OCF₃, and OSO₂R′; wherein R″ represents C₁₋₄ alkyl, or C₃₋₆ cycloalkyl.
Y is selected from the group consisting of C, CH, O, N and S; provided that when
Y is S, R₁^{IV} is O or not present; further provided that when Y is O, R₁^{IV} is not present;
X′ is selected from the group consisting of CH₂, NH, O and S.
R₉ is selected from the group consisting of O-alkyl, O—CH₂-aryl, and O-aryl;
X₁″″ is selected from the group consisting of CN, NO₂, Cl, F, Br, I, H, R, OR, CF₃, OCF₃ and OSO₂R, wherein R represents C₁₋₄ alkyl, or C₃₋₆cycloalkyl, X₂″″, X₃″″, and X₄″″ are, independently, selected from the group consisting of Cl, NO₂, Cl, F, Br, I, H, R′, OR′, CF₃, OCF₃ and OSO₂R′, provided that either X″″₁ or X″″₃ is H, wherein R′ is C₁₋₄ allyl or haloalkyl;
or X₁″″ and X₂″″ together form an aryl or heteroaryl ring, substituted or unsubstituted; wherein the heteroatom is selected from N, S and O and the substituents are selected from the group consisting of halo, C₁₋₄ alkyl, OCF₃, CF₃, OMe, CN, OSO₂-C₁₋₄ alkyl, OSO₂-C₃₋₆ cycloalkyl and NO₂;
or X₂″″ and X₄″″ independently represent C(O)R₁;
and pharmaceutically acceptable salts and complexes thereof.

2. A compound according to claim 1 having a structure according to Formula (II) hereinbelow:: 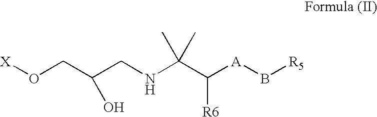
wherein:
R₅ is heteroaryl or fused heteroaryl; wherein the hetero-ring contains N, O or S, and is aromatic, dihydro or tetrahydro, unsubstituted or substituted with any substituents being selected from the group consisting of OH, OCH₃, CH(CH₃)₂, halogen, C₁₋₄ alkyl, C₁₋₄ alkoxy, C₃₋₆ cycloalkyl, OSO₂R^{IV}, CN, NO₂, OCF₃, CF₃, CH₂CF₃, (CH₂)ₙCO₂H, (CH₂)ₙCO₂R^{IV}, and O—(CH₂)ₙCO₂R^{IV}; and
A and B are, independently, selected from the group consisting of a bond, CH₂, NH, O, S and C═O, provided that either A or B is selected from CH₂ and NH; or A and B together form a bond; or the A-B moiety is represented by CH═CH or C≡C.

3. A compound according to claim 2 wherein:
R₅ is heteroaryl or fused heteroaryl, wherein the hetero-ring contains N, O or S and is aromatic, dihydro or tetrahydro, unsubstituted or substituted with any substituents being selected from the group consisting of OCH₃, halogen, C₁₋₄ alkyl, CN, NO₂, OCF₃, CF₃, and CH₂CF₃;
R₆ is H; and
A and B are, independently, selected from the group consisting of a bond, CH₂, NH, O, S and C═O, provided that either A or B is selected from CH₂ and bond of A and B together form a bond.

4. A compound according to claim 3 wherein.
R₅ is heteroaryl or fused heteroaryl, wherein the hetero-ring contains N, O or S and is aromatic, dihydro or tetrahydro, unsubstituted or substituted with any substituents being selected from the group consisting of OCH₃, halogen, C₁₋₄ alkyl, CN, NO₂, OCF₃, CF₃, and CH₂CF₃;
R₆ is H; and
A and B are, independently, selected from the group consisting of a bond, CH₂, O, or A and B together form a bond.

5. A compound according to claim 1 selected from the group consisting of:
(R)-N-[2-Hydroxy-3-(3-chloro-2-cyanophenoxy)propyl]-1,1-dimethyl-2-(2,3-dihydrobenzo[b]furan-5yl)ethylamine;
(R)-N-[2-Hydroxy-3-(3-chloro-2-cyanophenoxy)propyl]-1,1-dimethyl-2-(quinolin-3-yl)ethylamine;
(R)-N-[2-Hydroxy-3-(3-chloro-2-cyanophenoxy)propyl]-1,1-dimethyl-2-(quinolin-2-yl)ethylamine;
(R)-N-[2-Hydroxy-3-(3-chloro-2-cyanophenoxy)propyl]-1,1-dimethyl-2-(isoquinolin-3-yl)ethylamine;
(R)-N-[2-Hydroxy-3-(3-chloro-2-cyanophenoxy)propyl]-1,1-dimethyl-4-(2-pyridyl)butylamine;
(R)-N-[2-Hydroxy-3-(3-chloro-2-cyano-4-morpholinosulfonamidophenoxy)propyl]-1,1-dimethyl-4-(2-pyridyl)butylamine;
(R)-N-[2-Hydroxy-3-(3-chloro-2-cyanophenoxy)propyl]-1,1-dimethyl-4-(3-pyridyl)butylamine;
(R)-N-[2-Hydroxy-3-(3-chloro-2-cyano-4-morpholinosulfonamidophenoxy)propyl]-1,1-dimethyl-4-(3-pyridyl)butylamine;
(R)-N-[2-Hydroxy-3-(3-chloro-2-cyanophenoxy)propyl]-1,1-dimethyl-4-(4-carbethoxyphenyl)butylamine;
(R)-N-[2-Hydroxy-3-(3-chloro-2-cyanophenoxy)propyl]-1,1-dimethyl-2-(4-ethylpyrid-2-yl)ethylamine;
(R)-N-[2-Hydroxy-3-(3-chloro-2-cyanophenoxy)propyl]-1,1-dimethyl-2-benzamidoethylamine;
(R)-N-[2-Hydroxy-3-(3-chloro-2-cyanophenoxy)propyl]-4-phenylbutylamine,
(R)-N-[2-Hydroxy-3-(3-chloro-2-cyanophenoxy)propyl]-1,1-dimethyl-4-phenylbut-2-ynylamine;
and pharmaceutically acceptable salts and complexes thereof.

6. A compound according to claim 5 selected from the group consisting of:
(R)-N-[2-Hydroxy-3-(3-chloro-2-cyanophenoxy)propyl]-1,1-dimethyl-2-(2,3′-dihydrobenzo[b]furan-5yl)ethylamine;
(R)-N-[2-Hydroxy-3-(3-chloro-2-cyanophenoxy)propyl]-1,1-dimethyl-2-(quinolin-3-yl)ethylamine;
(R)-N-[2-Hydroxy-3-(3-chloro-2-cyanophenoxy)propyl]-1,1-dimethyl-2-(quinolin-2-yl)ethylamine;
(R)-N-[2-Hydroxy-3-(3-chloro-2-cyanophenoxy)propyl]-1,1-dimethyl-2-(isoquinolin-3-yl)ethylamine;
(R)-N-[2-Hydroxy-3-(3-chloro-2-cyanophenoxy)propyl]-1,1-dimethyl-4-(2-pyridyl)butylamine;
(R)-N-[2-Hydroxy-3-(3-chloro-2-cyano-4-morpholinosulfonamidophenoxy)propyl]-1,1-dimethyl-4-(2-pyridyl)butylamine;
(R)-N-[2-Hydroxy-3-(3-chloro-2-cyanophenoxy)propyl]-1,1-dimethyl-4-(3-pyridyl)butylamine;
(R)-N-[2-Hydroxy-3-(3-chloro-2-cyano-4-morpholinosulfonamidophenoxy)propyl]-1,1-dimethyl-4-(3-pyridyl)butylamine;
(R)-N-[2-Hydroxy-3-(3-chloro-2-cyanophenoxy)propyl]-1,1-dimethyl-2-(4-ethylpyrid-2-yl)ethylamine;
(R)-N-[2-Hydroxy-3-(3-chloro-2-cyanophenoxy)propyl]-1,1-dimethyl-2-benzamidoethylamine; and
(R)-N-[2-Hydroxy-3-(3-chloro-2-cyanophenoxy)propyl]-4-phenylbutylamine;
and pharmaceutically acceptable salts and complexes thereof.

7. A compound according to claim 6 selected from the group consisting of:
(R)-N-[2-Hydroxy-3-(3-chloro-2-cyanophenoxy)propyl]-1,1-dimethyl-2-(2,3-dihydrobenzo[b]furan-5yl)ethylamine;
(R)-N-[2-Hydroxy-3-(3-chloro-2-cyanophenoxy)propyl]-1,1-dimethyl-2-(quinolin-3-yl)ethylamine:
(R)-N-[2-Hydroxy-(3-chloro-2-cyanophenoxy)propyl]-1,1-dimethyl-2-(quinolin-yl)ethylamine;
(R)-N-[2-Hydroxy-(3-chloro-2-cyanophenoxy)propyl]-1,1-dimethyl-2-(isoquinolin-3-yl)ethylamine;
(R)-N-[2-Hydroxy-3-(3-chloro-2-cyanophenoxy)propyl]-1,1-dimethyl-4-(2-pyridyl)butylamine;
(R)-N-[2-Hydroxy-3-(3-chloro-2-cyano-4-morpholinosulfonamidophenoxy)propyl]-1,1-dimethyl-4-(2-pyridyl)butylamine,
(R)-N-[2-Hydroxy-3-(3-chloro-2-cyanophenoxy)propyl]-1,1-dimethyl-4-(3-pyridyl)butylamine;
(R)-N-[2-Hydroxy-3-(3-chloro-2-cyano-4-morpholinosulfonamidophenoxy)propyl]-1,1-dimethyl-4-(3-pyridyl)butylamine; and
(R)-N-[2-Hydroxy-3-(3-chloro-2-cyanophenoxy)propyl]-1,1-dimethyl-2-(4-ethylpyrid-2-yl)ethylamine;
and pharmaceutically acceptable salts and complexes thereof.

8. A pharmaceutical composition for use in treating a disease or disorder characterized by an abnormal bone or mineral homeostasis which comprises a compound according to claim 1 and a pharmaceutically acceptable carrier.

9. A method of antagonizing a calcium receptor which comprises administering to a subject in need thereof an effective amount of a compound according to claim 1.

10. A method of treating a disease or disorder characterized by an abnormal bone or mineral homeostasis which comprises administering to a subject in need of treatment an effective amount of a compound according to claim 1.

11. A method according to claim 10 wherein the bone or mineral disease or disorder is selected from the group consisting of osteosarcoma, periodontal disease, fracture healing, osteoarthritis, rheumatoid arthritis, Paget's disease, humoral hypercalcemia, malignancy and osteoporosis.

12. A method according to claim 11 wherein the bone or mineral disease or disorder is osteoporosis.

13. A method according of increasing serum parathyroid levels which comprises administering to a subject in need of treatment an effective amount of a compound according to claim 1.

14. Use of a compound according to claim 1 in the manufacture of a medicament for use in treating a disease or disorder characterized by an abnormal bone or mineral homeostasis.
